(19) United States
(12) Patent Application Publication
(10) Pub. No.: US 2017/0360843 A1
(43) Pub. Date: Dec. 21, 2017

(54) METHODS FOR THE TREATMENT OF POLYCYSTIC OVARY SYNDROME

(71) Applicants: INSTITUTE OF ZOOLOGY, CHINESE ACADEMY OF SCIENCES, Beijing (CN); CENTER FOR REPRODUCITIVE MEDICINE, SHANDONG PROVINCIAL HOSPITAL, SHANDONG UNIVERSITY, Jinan (CN)

(72) Inventors: Xiaoxue YUAN, Beijing (CN); Tao HU, Beijing (CN); Yuanyuan HUANG, Beijing (CN); Wanzhu JIN, Beijing (CN); Zi-Jiang CHEN, Broadview Heights, OH (US)

(21) Appl. No.: 15/185,517

(22) Filed: Jun. 17, 2016

(51) Int. Cl. A61K 35/35; A61K 38/22; A61K 38/19

(52) U.S. Cl. CPC A61K 35/35; A61K 38/19; A61K 38/22

(57) ABSTRACT

Polycystic ovary syndrome (PCOS) is a complex and heterogeneous endocrine syndrome associated with a high risk for the development of insulin resistance, type 2 diabetes (T2D), obesity, dyslipidemia, and cardiovascular disease. There are no known therapies for the 9-18% of women afflicted with PCOS. Provided herein are methods for the treatment and/or prevention of at least one symptom of PCOS. Also provided herein are methods for the treatment and/or prevention of PCOS.

### FIELD

The present disclosure relates to the field of therapeutic interventions, and particularly to therapeutic interventions for polycystic ovary syndrome and related conditions.

### BACKGROUND

Polycystic ovary syndrome (PCOS) is a common endocrine system disorder affecting women of reproductive age. The disorder is characterized by an imbalance in the sex hormones estrogen and progesterone. Women with PCOS may have enlarged ovaries that contain small collections of fluid—called follicles—located in each ovary as seen during an ultrasound exam. The exact cause of polycystic ovary syndrome is unknown. Early diagnosis and treatment along with weight loss may reduce the risk of long-term complications.

More specifically, PCOS is a complex endocrinopathy and is one of the most common endocrine diseases in women of reproductive age. While not easily quantified, it is believed that from 9% to 18% of women are affected during their lifetime. The core features of PCOS include polycystic ovaries, hyperandrogenism, and chronic anovulation.

PCOS is a complex and heterogeneous syndrome associated with a high risk for the development of insulin resistance, type 2 diabetes (T2D), obesity, dyslipidemia, and cardiovascular disease. There are three different criteria generally used for the diagnosis of PCOS: androgen excess, irregular menstruation, and polycystic ovary appearance on ultrasound after excluding other causes of hyperandrogenism and anovulation. Because a single etiologic factor is not able to fully account for all of the clinical features in PCOS, the pathogenesis of PCOS is largely unknown. Several genetic and environmental factors may contribute to the development of PCOS; however, the underlying cellular mechanism of the induction and progression of PCOS remains to be identified.

Insulin resistance, which is common among PCOS patients, seems to be a key etiological characteristic, and about 85% of women with PCOS suffer from insulin resistance. Compensatory hyperinsulinemia can directly stimulate ovarian and adrenal secretion of androgen and decrease hepatic sex hormone binding globulin (SHBG) synthesis, resulting in an increased bioavailability of free testosterone levels. Thus, insulin resistance and hyperandrogenism contribute to the key clinical presentation of PCOS. Because the clinical features are complex and vary among PCOS patients, it is hard to provide a first-line treatment for PCOS. Most treatment guidelines recommend that patients change lifestyles, including exercise and dietary modification to combat specific symptoms. Patients can take oral contraceptive pills to control symptoms of hyperandrogenism or take insulin-sensitizing medicines such as metformin or pioglitazone when they have impaired glucose tolerance or features of a metabolic syndrome. However, there is a lack of effective treatment for PCOS in general at present.

### SUMMARY

The present disclosure provides methods for treating, delaying the progression, and/or preventing PCOS, methods for treating, delaying the progression, and/or preventing PCOS associated conditions, and methods for treating, delaying the progression, and/or preventing at least one symptom of PCOS.

In an exemplary embodiment, a method for the treatment, delay of progression, or prevention of at least one symptom of polycyctic ovary syndrome is provided. The method comprises increasing brown adipose tissue activity in a subject in need thereof, wherein at least one symptom of polycystic ovary syndrome is reduced.

In an exemplary embodiment a method for treating, delay the progression, or preventing PCOS is provided. The method comprises increasing brown adipose tissue activity in a subject in need thereof, wherein at least one symptom of polycystic ovary syndrome is reduced.

In an exemplary embodiment a method for treating, delaying the progression, or preventing PCOS is provided. The method comprises administration of adiponectin to a subject in need thereof, wherein at least one symptom of polycystic ovary syndrome is reduced.

### BRIEF DESCRIPTION OF THE DRAWINGS

Several technical aspects of the present disclosure will be described in more detail below with reference to the accompanying drawings in order for those skilled in the art to be able to carry out the present disclosure. The present disclosure may, however, be embodied in many different forms and should not be construed as limited to the exemplary embodiments set forth herein. In particular, the general inventive concepts are not intended to be limited by the various illustrative embodiments disclosed herein.

FIG. 1 shows the results of several tests demonstrating that BAT transplantation reverses PCOS BAT activity: (A) shows a PET-CT, (B) shows activity of brown adipose tissue, expressed as the standard uptake values (SUVs), (C) shows BAT-specific marker gene expression, (D) shows OXPHOS protein expression, and (E) shows UCP1 expression.

FIG. 2 shows the effects of BAT transplantation on body weight and food intake: (A) shows food intake and (B) body weight (n=8-10 per group).

FIG. 3 shows the results of BAT transplantation on metabolic abnormality: (A) shows an infrared thermal image, (B) shows body temperature, (C) shows whole body energy expenditure, (D) shows a bar graph of energy expenditure, (E) shows glucose tolerance, and (F) shows a glucose tolerance test after DHEA induced insulin intolerance.

FIG. 4 shows the results of BAT transplantation on metabolic abnormality: (A) shows glucose tolerance test (GTT), (B) shows insulin tolerance test (ITT), and (C) shows insulin levels during GTT

FIG. 5 shows the results of BAT transplantation on PCOS acyclicity, ovarian phenotypes, and infertility: (A) shows estrous cycles in control and treated subjects, (B) shows concentrations of follicle stimulating hormone (FSH) and luteinizing hormone (LH) levels, (C) shows the LH/FSH ratio, (D) shows histological results from ovarian samples (cystic follicles indicated with arrow), (E) shows the expression of ovarian steroidogenic enzymes, and (F) demonstrates ability of treated subjects to produce a litter.

FIG. 6 shows the results of various tests after BAT transplantation: (A) shows endogenous BAT activity as evidenced by PET-CT scan, (B) shows endogenous BAT activity, (C) shows infrared thermal images demonstrating that adiponectin treatment significantly reversed DHEA mediated body temperature reduction, (D) shows the results of body temperature reduction in graph form, (E) shows results of a glucose tolerance test, (F) shows results of insulin resistance (inner graph indicating area under the curve of GTT and ITT, respectively).

FIG. 7 shows the results of adiponectin administration on PCOS acyclicity, ovarian phenotypes, and infertility: (A) shows the concentrations of follicle stimulating hormone and luteinizing hormone (LH), (B) shows the LH/FSH ratio, (C) shows results of adiponectin administration on DHEA-induced acyclicity, (D) shows effects of adiponectin administration on pregnant capacity in the PCOS subject.

FIG. 8 shows the results of adiponectin administration on energy expenditure: (A) shows circulating adiponectin levels in human subjects compared with controls, (B) shows circulating adiponectin levels in rodent subjects compared with controls (Data were analyzed by unpaired t test in A. n=40 per group. **P<0.01 versus control or analyzed by one-way ANOVA with Tukey's post hoc test in B. n=8-10 per group. Different characters indicate P<0.05), (C) shows oxygen consumption, (D) shows oxygen consumption in graph form (Data were analyzed by one-way ANOVA with Tukey's post hoc test. n=6 per group. Different lowercase letters indicate significant differences among groups (One-way ANOVA, with Tukey's post hoc test, P<0.05).

### DETAILED DESCRIPTION

The present invention and associated general inventive concepts will be further described hereinafter in detail with reference to the accompanying drawings and various exemplary embodiments. One of ordinary skill in the art will appreciate that these exemplary embodiments only constitute a fraction of the possible embodiments encompassed by the present invention and associated general inventive concepts. As such, the scope of the present disclosure is by no means limited to the exemplary embodiments set forth herein.

The terms “symptom of polycyctic ovary syndrome” or “polycystic ovary syndrome associated symptom,” as used herein, are used interchangeably, and refer to at least one of an imbalance in the sex hormones estrogen and progesterone, androgen excess, obesity, dyslipidemia, reduced energy expenditure, irregular menstruation, anovulation, insulin resistance, polycystic ovary appearance on ultrasound, and enlarged ovaries.

The term “polycystic ovary syndrome associated condition,” as used herein, refers to at least one of metabolic syndrome, hyperandrogenism, chronic anovulation, insulin resistance, type 2 diabetes (T2D), obesity, dyslipidemia, impaired glucose tolerance, and cardiovascular disease.

The term “regulation,” as used herein, refers to the targeted movement of at least one biological marker or variable associated with a subject condition.

The term “in need thereof,” “susceptible to,” and “at risk of,” as used herein, are used interchangeably to refer to individuals having little resistance to a certain condition or disease, including being genetically predisposed, having a family history of, and/or having symptoms of the condition or disease. In certain exemplary embodiments, the terms refer to a subject displaying at least one symptom of PCOS or a PCOS associated condition. The terms are intended to refer to an individual with a greater need or are at an increased risk as compared to the general population or subset thereof.

The methods of the present disclosure may comprise, consist of, or consist essentially of the essential elements of the methods as described herein, as well as any additional or optional element described herein or otherwise useful in therapeutic or other medical applications.

To the extent that the terms “includes,” “including,” “contains,” or “containing” are used in the specification or the claims, they are intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. Also, to the extent that the terms “in” or “into” are used in the specification or the claims, it is intended to additionally mean “on” or “onto.”

PCOS is a complex and heterogeneous syndrome associated with a high risk for the development of a variety of serious conditions. These PCOS associated conditions include insulin resistance, type 2 diabetes (T2D), obesity, dyslipidemia, cardiovascular disease, and metabolic syndrome, among others (See Yuan et al., PNAS Early Edition (2016), 10.1073, 1523236113, the content of which is incorporated by reference as if fully recited herein). There are three different criteria generally used for the diagnosis of PCOS: androgen excess, irregular menstruation, and polycystic ovary appearance on ultrasound after excluding other causes of hyperandrogenism and anovulation. Because a single etiologic factor is not able to fully account for all of the clinical features in PCOS, the pathogenesis of PCOS is largely unknown. Several genetic and environmental factors may contribute to the development of PCOS; however, the underlying cellular mechanism of the induction and progression of PCOS remains to be identified.

Insulin resistance, which is common among PCOS patients, seems to be a key etiological characteristic, and about 85% of women with PCOS suffer from insulin resistance. Compensatory hyperinsulinemia can directly stimulate ovarian and adrenal secretion of androgen and decrease hepatic sex hormone binding globulin (SHBG) synthesis, resulting in an increased bioavailability of free testosterone levels. Thus, insulin resistance and hyperandrogenism contribute to the key clinical presentation of PCOS. Because the clinical features are complex and vary among PCOS patients, it is hard to provide first-line treatment of PCOS.

In humans and other mammals, there are mainly two types of adipose tissue with opposing functions: white adipose tissue (WAT) and brown adipose tissue (BAT). The main function of WAT is to store excess energy in WAT as a form of triglycerides whereas BAT contains large numbers of mitochondria that uncouple large amounts of fuel for heat generation and the maintenance of body temperature. Generally, the ratio of WAT to BAT increases with age. However, recent studies using positron emission tomography (PET) have demonstrated that human adults also possess metabolically active BAT and that BAT activation (activity) inversely correlates with age and body mass index (BMI).

It has been demonstrated that BAT activity was dramatically reduced in a dehydroepiandrosterone (DHEA) (a precursor of androgen)-induced PCOS rat compared with a normal control rat. However, key features of PCOS, such as insulin resistance, irregular estrous cycle, and low birth rate, were significantly improved after BAT transplantation in PCOS rats. Interestingly, as shown herein, transplanted BAT in PCOS rats enhanced endogenous BAT activity and thereby increased the circulating adiponectin level, which was lower in both PCOS patients and PCOS rats. In parallel, exogenous adiponectin protein administration in a PCOS rat recapitulated the effects that were seen in a BAT transplanted PCOS rat. Exemplary embodiments demonstrate that BAT is an important organ regulating the features of PCOS and that an increase of BAT mass or its activity provides a therapy for treating, delaying the progression, or preventing a symptom of PCOS, a PCOS associated condition, and/or PCOS.

In certain exemplary embodiments, the present disclosure is directed to a method of treating, delaying the progression, or preventing a symptom of PCOS, a PCOS associated condition, and/or PCOS. Treating or preventing is intended to refer generally to an improvement or regulation of a symptom or indicator of a disease or condition commensurate with a therapeutic intervention. Thus, in certain exemplary embodiments, treatment or prevention refers to a movement of a selected symptom relative to a level of the same symptoms or indicators toward what would be expected for a subject who is not in need of therapy, but that is improved relative to that of an otherwise untreated subject.

In certain exemplary embodiments, the present disclosure is directed to a method for the prevention, delay of progression, or the treatment of a symptom of PCOS, a PCOS associated condition, and/or PCOS. The method comprises increasing a level of BAT activity or increasing an amount of BAT in a subject in need thereof. In certain exemplary embodiments, the subject to whom the BAT is provided is a subject displaying at least one symptom of PCOS or a PCOS associated condition. In certain exemplary embodiments, the subject has experienced, or is at risk of experiencing at least one symptom of PCOS or a PCOS associated condition.

In certain exemplary embodiments, increasing an amount of BAT activity according to the general inventive concepts provides an improvement in a marker or indicator associated with at least one symptom of PCOS or a PCOS associated condition. In particular, increasing BAT activity in a subject in need thereof according to the general inventive concepts results in an increase in circulating adiponectin level in the subject. The increased circulating adiponectin level may also lead to the prevention, delay of progression, or the treatment of a at least one symptom of PCOS or a PCOS associated condition. In particular, increasing BAT activity in a subject in need thereof according to the general inventive concepts results in an improvement in glucose tolerance in the subject. In particular, increasing BAT activity in a subject in need thereof according to the general inventive concepts results in an increase in energy expenditure in the subject. In particular, increasing BAT activity in a subject in need thereof according to the general inventive concepts results in an improvement in at least one of an imbalance in the sex hormones estrogen and progesterone, androgen excess, irregular menstruation, anovulation, insulin resistance, polycystic ovary appearance on ultrasound, enlarged ovaries, and obesity, and cardiovascular disease in the subject.

In certain exemplary embodiments, a reduction in one or more symptoms of PCOS in an individual, is identified by measuring the amount of a biomarker associated with PCOS or a PCOS related condition. The biomarker may be measured in a model organism following performance of a claimed method disclosed herein to the model organism. The model organism can be any known model organism for measuring these properties. In some embodiments, the model organism is a rodent.

In certain exemplary embodiments, BAT activity in a subject is increased. An increase in BAT activity may include an improvement in BAT activity to a level that is determined to be basal or normal when compared to a group of control subjects that do not have PCOS or a PCOS associated condition. In certain exemplary embodiments, the control subjects are otherwise healthy female subjects. In certain exemplary embodiments, an increase in BAT activity is measured relative to the individual subject an may include an increase in BAT activity of 1% to 100% or more, when compared to the BAT activity prior to beginning a therapeutic regimen. In certain exemplary embodiments, the subject's BAT activity is increased by at least 1%, including at least 2%, including at least 3%, including at least 4%, including at least 5%, including at least 6%, including at least 7%, including at least 8%, including at least 9%, including at least 10%, including at least 20%, including at least 30%, including at least 40%, including at least 50%, including at least 60%, including at least 70%, including at least 80%, including at least 90%, including 100% or more. In certain exemplary embodiments, the BAT activity of the subject is increased by more than 100%.

In certain exemplary embodiments, a level of circulating adiponectin in a subject is increased. An increase in a level of circulating adiponectin may include an improvement in circulating adiponectin to a level that is determined to be basal or normal when compared to a group of control subjects that do not have PCOS or a PCOS associated condition. In certain exemplary embodiments, the control subjects are otherwise healthy subjects. In certain exemplary embodiments, an increase in a level of circulating adiponectin is measured relative to the individual subject an may include an increase in the level of circulating adiponectin of 1% to 100% or more, when compared to the level of circulating adiponectin prior to beginning a therapeutic regimen. In certain exemplary embodiments, the subject's level of circulating adiponectin is increased by at least 1%, including at least 2%, including at least 3%, including at least 4%, including at least 5%, including at least 6%, including at least 7%, including at least 8%, including at least 9%, including at least 10%, including at least 20%, including at least 30%, including at least 40%, including at least 50%, including at least 60%, including at least 70%, including at least 80%, including at least 90%, including 100% or more. In certain exemplary embodiments, the level of circulating adiponectin of the subject is increased by more than 100%.

In certain exemplary embodiments, a symptom (or prevalence of said symptom) of PCOS is reduced. A reduction in the level of a symptom may include an improvement to a level of the particular symptom that is determined to be basal or normal when compared to a group of control subjects that do not have PCOS or a PCOS associated condition. In certain exemplary embodiments, the control subjects are otherwise healthy female subjects. In certain exemplary embodiments, a reduction in a particular symptom is measured relative to the individual subject an may include an reduction of 1% to 100%, when compared to the particular symptom to beginning a therapeutic regimen. In certain exemplary embodiments, the subject's symptom is reduced by at least 1%, including at least 2%, including at least 3%, including at least 4%, including at least 5%, including at least 6%, including at least 7%, including at least 8%, including at least 9%, including at least 10%, including at least 20%, including at least 30%, including at least 40%, including at least 50%, including at least 60%, including at least 70%, including at least 80%, including at least 90%, including 100% or more. In certain exemplary embodiments, the particular symptom displayed by the subject is reduced by 100%.

### Examples

All animal studies were conducted with the approval of the Institutional Animal Care and Use Committee of the Institute of Zoology, Chinese Academy of Sciences. Tissue (0.5 g of BAT or muscle) transplantation experiments were performed in a DHEA-induced PCOS rat. Recombinant adiponectin [10 μg/kg body weight (BW)] was daily injected into a PCOS rat. Written informed consent was obtained from all human participants and this study was approved by the Institutional Review Board of Reproductive Medicine of Shandong University.

Human Subjects. All participants in the study were recruited from the Center for Reproductive Medicine, Shandong Provincial Hospital Affiliated with Shandong University, during the period from September 2014 to March 2015. Anthropometric variables, such as age, body mass index (BMI), menstrual cycle, and select endocrine and biochemical parameters, were recorded and are shown in Table 3.

The diagnosis of PCOS was based on the following revised Rotterdam diagnostic criteria for PCOS, which require the presence of at least two of the following: (i) oligoovulation and/or anovulation; (ii) clinical and/or biochemical signs of hyperandrogenism; and (iii) polycystic ovaries. Diagnoses of PCOS were made after exclusion of other etiologies for hyperandrogenemia and ovulatory dysfunction (e.g., 21-hydroxylase deficiency, congenital adrenal hyperplasia, Cushing syndrome, androgen-secreting tumors, thyroid disease, and hyperprolactinemia). All subjects in the control group had regular menstrual cycles and normal ovarian morphology, and total testosterone was evaluated to exclude hyperandrogenism. Peripheral blood samples were collected from all subjects during days 2-4 of spontaneous cycles after an overnight fast. Follicle-stimulating hormone (FSH), luteinizing hormone (LH), and testosterone were tested by chemiluminescence immunization (Beckman Access Health Company). The hematological biochemical variables were measured at fasting. A 75-g oral glucose tolerance test (OGTT) was carried out, and the plasma glucose and serum insulin at fasting and 2-h postload were measured. The serum was centrifugally separated from procoagulant peripheral blood. Written informed consent was obtained from all participants and this study was approved by the Institutional Review Board of Reproductive Medicine of Shandong University.

Animals. Female and male Sprague-Dawley rats (3 wk old) were purchased from Vital River Laboratory Animal Technology Co Ltd. Five rats per cage were housed under constant environmental conditions in the Office of Laboratory Animal Welfare-certified animal facility with a 12-h light-dark cycle. Food and water were provided ad libitum. All animal studies were conducted with the approval of the Institutional Animal Care and Use Committee of the Institute of Zoology, Chinese Academy of Sciences.

Establishment of the PCOS Model and Assessment of the Estrous Cycle. The DHEA used for establishing the PCOS model was purchased from Yangzhou Pharmaceutical Co., Ltd (cat. no. H10940064). From 4 wk of age, female rats were injected daily (s.c.) with DHEA (6 mg/100 g body weight) dissolved in 0.2 mL of PBS for 20 consecutive days. Control rats were injected with 0.2 mL of PBS for an equivalent length of time. The successful PCOS rat model was selected according to the known criteria, which was assessment of estrous cycle by vaginal cytology for eight consecutive days, together with a glucose tolerance test (GTT) for the following experiment.

Tissue Transplantation. The experiment was conducted according to the methods described previously. The operation was carried out in sterile conditions. Age- and sex-matched donor and recipient rats were used for the tissue transplantation experiment. BAT and part of the quadriceps were harvested from the donor rats, which were anesthetized with avertin (400 mg/kg body weight i.p.), and were placed in sterile saline. The recipient rats were anesthetized with avertin (400 mg/kg body weight i.p.), and the donor tissues (0.5 g for each recipient rat) were transplanted into the s.c. space of the dorsal region adjacent to the endogenous BAT as quickly as possible. The sham-operated rats underwent the same procedure, except receiving donor tissues.

Adiponectin Protein Treatment. Recombinant adiponectin protein (50636-MO8H-100) was purchased from Sino Biological Inc. PCOS rats were treated with exogenous recombinant adiponectin protein (10 μg/kg body weight/day i.p.) once a day for 20 consecutive days. The control group was treated with sterile PBS.

Glucose Tolerance Test and Insulin Tolerance Test. For glucose tolerance tests (GTTs), female rats were fasted for 16 h (1700 to 0900), with free access to drinking water, and injected with D-glucose (2.0 g/kg body weight) intraperitoneally. Blood glucose level was measured before and 15, 30, 60, 90, and 120 min after i.p. glucose injection by using an Accu-Chek glucose monitor (Roche Diagnostics Corp.). For the insulin tolerance test (ITT), female rats were fasted for 4 h (0900 to 1300), with free access to drinking water, and injected with insulin (1 U/kg body weight) (Humulin; Eli Lilly) intraperitoneally. Blood glucose levels were measured before and 15, 30, and 60 min after insulin injection.

Resting Metabolic Rate. The female rats were housed with one rat per cage, with free access to food and water. Metabolic rate was determined by oxygen consumption measurement performed for two consecutive days using a TSE laboratory master system as previously described.

Infrared Thermography and Core Temperature. Rats were exposed to a cold chamber (4° C.) with one rat per cage for up to 4 h, with free access to food and water. Images were taken using an infrared digital thermographic camera (E60: Compact Infrared Thermal Imaging Camera; FLIR) and were analyzed using FLIR Quick Report software (FLIR ResearchlR Max 3.4; FLIR). Core body temperature was measured using a rectal probe connected to a digital thermometer (Yellow Spring Instruments).

Micro PET/CT. PET/CT imaging was achieved with the Siemens Inveon Dedicated PET (dPET) System and Inveon Multimodality (MM) System (CT/SPECT) (Siemens Preclinical Solutions) at the Institute of Laboratory Animal Sciences, Chinese Academy of Medical Sciences. Mice were allowed to fast overnight and were lightly anesthetized with isoflurane, followed by a tail vein injection of 18 F-FDG (500 mCi). Mice were subjected to PET/CT analysis at 60 min after radiotracer injection. Inveon Acquisition Workplace (IAW) software was used for the scanning process. A 10-min CT X-ray for attenuation correction was scanned with a power of 80 kV and 500 μA and an exposure time of 1,100 ms before PET scan. Ten-minute static PET scans were acquired, and images were reconstructed by an OSEM3D algorithm followed by Maximization/Maximum a Posteriori (MAP) or FastMAP provided by IAW. The 3D regions of interest (ROIs) were drawn over the guided CT images, and the tracer uptake was measured using the software of Inveon Research Workplace (IRW) (Siemens). Individual quantification of the 18 F-FDG uptake in each of the ROIs was calculated. The data for the accumulation of 18 F-FDG on micro PET images were expressed as the standard uptake values (SUVs), which were determined by dividing the relevant ROI concentration by the ratio of the injected activity to the body weight. The data are presented as the mean±SEM.

Fertility Assessment. To examine fertility, female rats mated with proven stud males. Next day, successful mating was judged by observation of a vaginal plug. After 10 d, the few female rats were killed and were examined at implantation sites to confirm pregnancy. The rest of the animals were allowed to undergo natural delivery to produce pups.

Blood Analysis. The blood samples were collected by cardiac exsanguination under Avertin anesthesia, and the plasma samples were frozen and stored at 80° C. until further analysis. Rat plasma levels of LH, FSH, and insulin were analyzed using ELISA kits (NanJing Jian Cheng Bioengineering Institute). The plasma level of adiponectin in both rat and human samples was analyzed using ELISA kits (R&D Systems).

Gene Expression Analysis. Total RNA was isolated using the Rneasy Mini Kit. The cDNA was synthesized using random hexamers (Invitrogen) for subsequent real-time quantitative PCR analysis (ABI Prism VIIA7; Applied Biosystems). PCR products were detected using SYBR Green and normalized by cyclophilin expression. Primers were designed using Primer Quest (Integrated DNA Technologies).

Western Blot Analysis. Tissues were dissolved in RIPA buffer (150 mM sodium chloride, 1.0% Triton X-100, 0.5% sodium deoxycholate, 0.1% SDS, 50 mM Tris, protease and phosphatase inhibitor mixture (Roche Diagnostics). Protein concentrations were determined using a BCA assay kit (Pierce Diagnostics). Protein was separated by 10% (wt/vol) SDS/PAGE, transferred to a PVDF membrane (Millipore), blocked in 5% (wt/vol) skim milk in TBST (0.02 M Tris base, 0.14 M NaCl, 0.1% Tween 20, pH 7.4), and incubated with primary antibodies overnight at 4° C. and then incubated with secondary antibodies conjugated with HRP. The following primary antibodies were used: anti-UCP1 (1:1,000; Abcam), anti-OXPHOS (1:250; Abcam), and anti-GAPDH (1:1,000; Cell Signaling Technology). Signals were detected with super signal west pico-chemiluminescent substrate (Pierce).

Histology and Immunohistochemistry Analysis. Tissues were fixed in 4% paraformaldehyde overnight at room temperature and then embedded in paraffin. Sections of 5 μm thickness were stained with hematoxylin and eosin (H&E), and then images were taken by microscope (DS-RI1; Nikon). The number of antral follicles (600-1,000 μm in diameter) and corpora lutea were counted based on morphology and diameter. The thickness of theca cell layers was measured using ImageJ software (version 1.48; NIH) (n=6 per group, serial sections of each ovary were used for measurement). The standard streptavidin-biotin-peroxide immunostaining procedure was used for the detection of tyrosine hydroxylase (TH). Tissue specimens were blocked with 10% normal goat serum for 60 min and then incubated with anti-UCP1 (1:400 dilution; Santa Cruz Biotechnologies) or anti-TH (1:400 dilution; Pel FreeZ) antibody overnight at 4° C., followed by a 1-h incubation with HRP-conjugated goat anti-rabbit IgG at room temperature.

Statistical Analysis. Comparisons between groups were made by one-way ANOVA with Tukey's post hoc test or Student's t tests. A difference between groups of P<0.05 was considered significant.

### Discussion

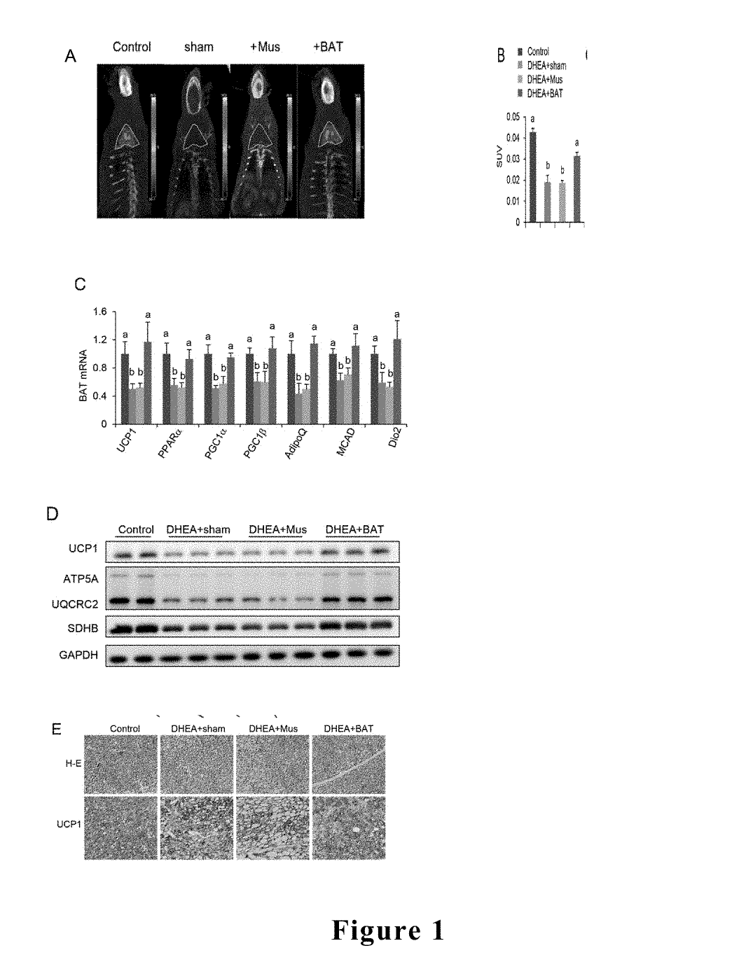

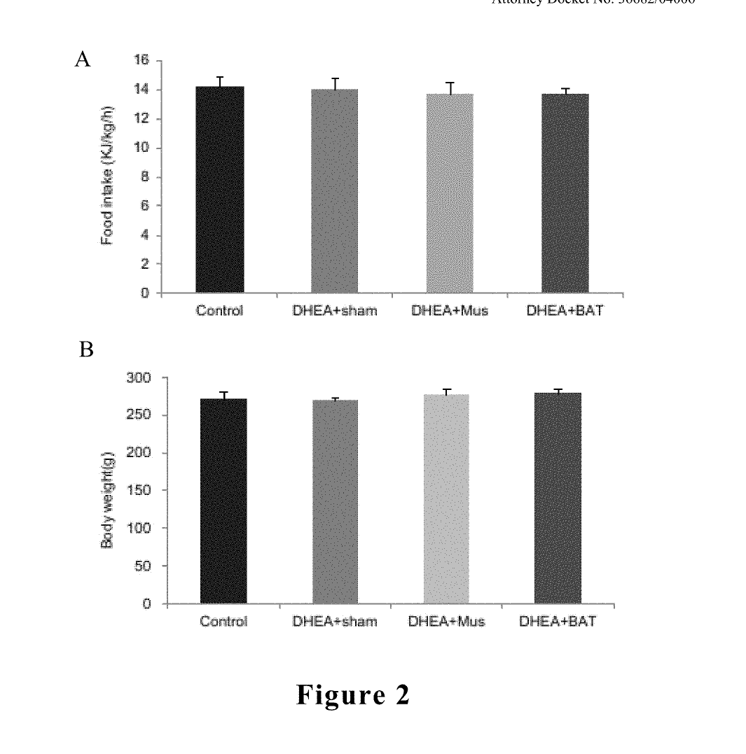

Exemplary embodiments demonstrate that increasing the amount of BAT (e.g., by transplantation) reverses reduced BAT activity and metabolic abnormality in certain subjects (e.g., subjects displaying at least one symptom of PCOS). Accumulating evidence indicates that insulin resistance is one of the most common clinical features in PCOS and that insulin resistance is often accompanied with reduced BAT activity. In certain examples described herein, a rat was injected with DHEA daily for 20 d and then the irregular estrous cycle was analyzed by vaginal smear check to confirm the development of PCOS. Next, 0.5 g of BAT was transplanted from an age- and sexmatched donor rat into a PCOS rat (DHEA+BAT), and three other groups—a PBS-treated (control) group, a sham-operated (DHEA+sham) group, or a skeletal muscle-transplanted (DHEA+Mus) group—served as control groups. At 3 wk after tissue transplantation, BAT activity was assessed with positron emission tomography-computed tomography (PET-CT). Results from PET-CT showed that BAT activity was significantly reduced in DHEA+sham and DHEA+Mus groups than in the control group; BAT transplantation into a DHEA-induced PCOS rat dramatically increased endogenous BAT activity up to the level of the control group (FIGS. 1 A and B). Although obesity is a key feature of PCOS, there was no significant difference of body weight as well as food consumption among groups in the current study (FIG. 2). Uncoupling protein 1 (UCP1) is a BAT-specific protein that dissipates the proton electrochemical gradient in mitochondria to generate heat. Peroxisome proliferator activated receptor gamma coactivator 1 alpha (PGC1α) and peroxisome proliferator activated receptor gamma coactivator 1 beta (PGC1β) induce the expression of UCP1 and mitochondria thermogenesis-related genes. Peroxisome proliferator activated receptor alpha (PPARα) is a major regulator of lipid metabolism. Type II iodothyronine deiodinase (Dio2) is a marker gene of BAT activation. Therefore, we analyzed the gene expression levels of these genes to assess BAT thermogenic activity. In parallel to BAT activity results, BAT-specific gene expressions were significantly decreased in DHEA+sham and DHEA+Mus groups compared with control and DHEA+BAT groups (FIG. 1C).

Moreover, UCP1 and OXPHOS protein expressions were also increased in the DHEA+BAT group compared with DHEA+sham or DHEA+Mus groups (FIGS. 1 D and E).

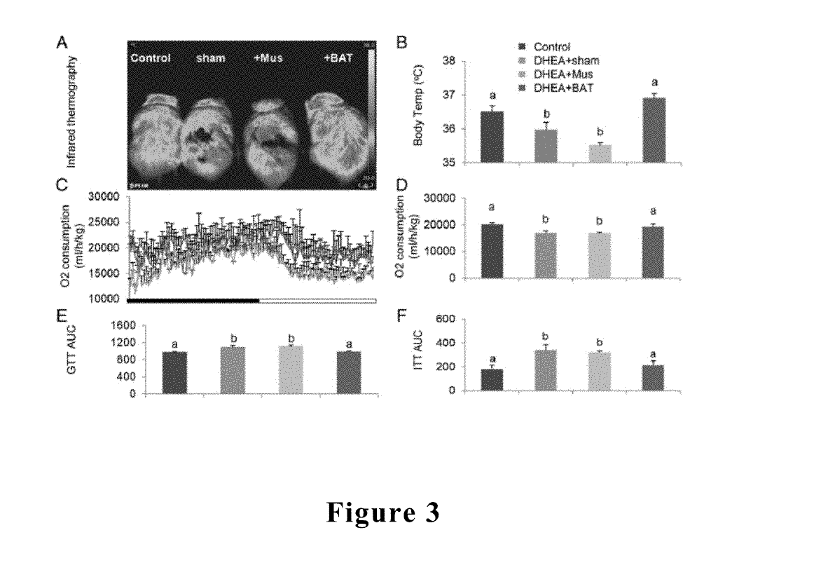

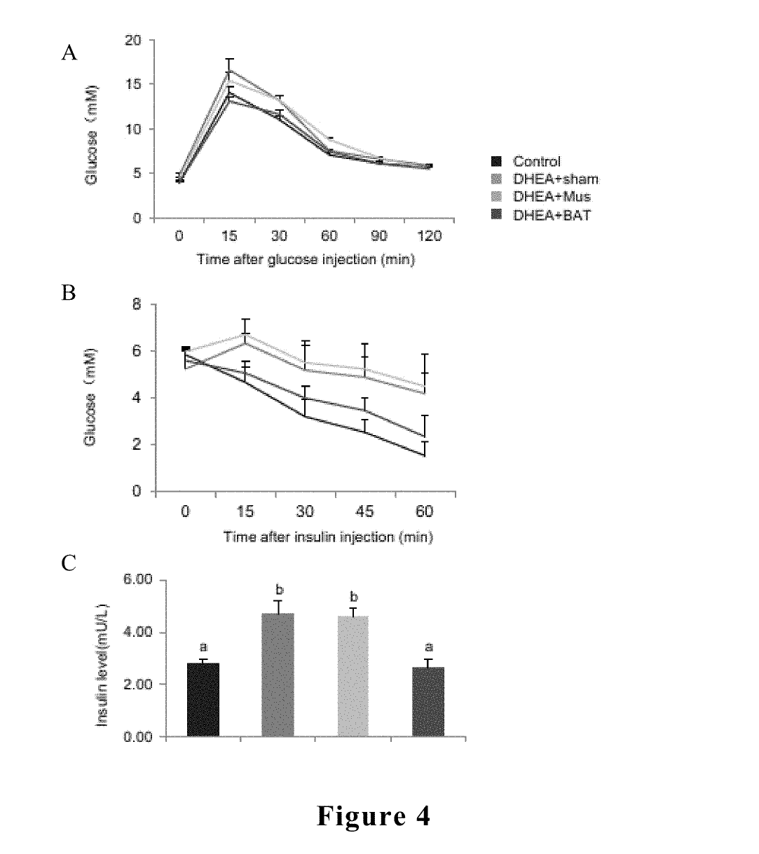

As shown herein, body temperature after cold exposure was significantly decreased in DHEA+sham and DHEA+Mus groups whereas BAT transplantation significantly reversed DHEA mediated body temperature reduction (FIGS. 3 A and B). In addition, BAT transplantation, not sham operation or skeletal muscle transplantation, significantly improved energy expenditure in a DHEA-induced PCOS rat (FIGS. 3 C and D). Consequently, glucose homeostasis and insulin sensitivity were dramatically improved in the DHEA+BAT group compared with DHEA+sham or DHEA+Mus groups (FIGS. 3 E and F and FIG. 4). BAT transplantation reverses DHEA-induced glucose intolerance as evidenced by (FIG. 4 A) glucose tolerance test (GTT) and (FIG. 4 B) insulin tolerance test (ITT), as well as insulin levels during GTT (C). Data were analyzed by one-way ANOVA with Tukey's post hoc test. n=8-10 per group. Different lowercase letters indicate significant differences among groups (One-way ANOVA, with Tukey's post hoc test, P<0.05). These results suggest that BAT transplantation reverses (i.e., improves) endogenous BAT activity and insulin resistance in the DHEA-induced PCOS rat.

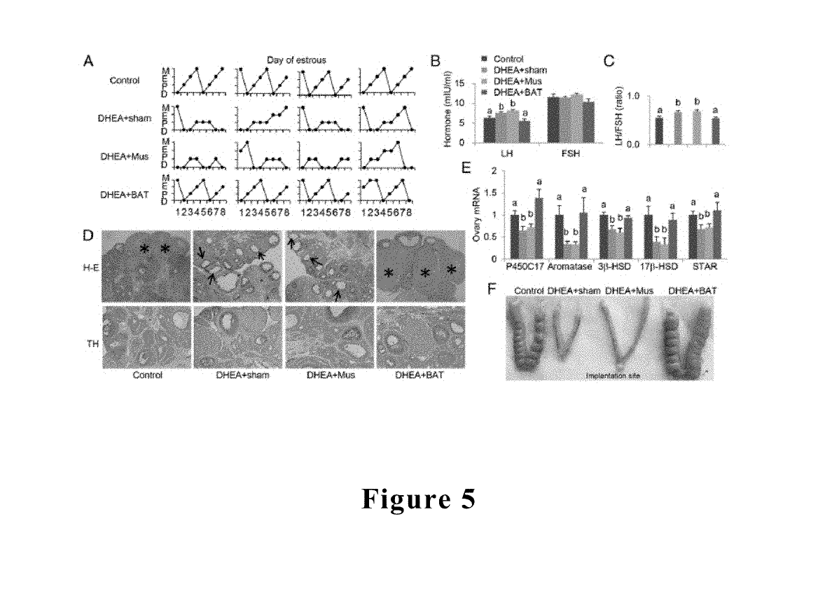

In certain exemplary embodiments, increasing a level of BAT activity (e.g., by administration of adiponectin) or increasing an amount of BAT in a subject (e.g., by BAT transplantation) reverses PCOS acyclicity. As mentioned above, irregular menstruation is one of the key criteria for the diagnosis of PCOS. After DHEA treatment, acyclicity detected by vaginal cytology was found in the DHEA+sham group and not in the control group, indicating that a rat PCOS model had been successfully developed (FIG. 5A and Table 1). Surprisingly, BAT transplantation normalized menstrual cyclicity in 7 out of 10 DHEA-induced PCOS rats, which was not found in the DHEA+Mus group.

TABLE 1

| Group | Total No. | Normal Estrous Cycle | Abnormal Cycle |
|---|---|---|---|
| Control | 8 | 8 | 0 |
| DHEA + sham | 9 | 1 | 8 |
| DHEA + Mus | 9 | 2 | 7 |
| DHEA + BAT | 10 | 7 | 3 |

These results highlight that BAT transplantation and/or increasing an amount of BAT activity reverses abnormal estrous cycles in the PCOS rat. Abnormal estrous is accompanied with altered plasma gonadotropin concentration. Although plasma follicle-stimulating hormone (FSH) concentration was not altered among groups, plasma-luteinizing hormone (LH), as well as the LH/FSH ratio, which is one of the parameters for the diagnosis of PCOS in clinics, was significantly increased in DHEA+sham and DHEA+Mus groups compared with the control group. Notably, BAT transplantation reversed the plasma LH level and LH/FSH ratio to a normal level (FIGS. 5 B and C). Additionally, the plasma testosterone (T) level was significantly attenuated after BAT transplantation in a DHEA-induced PCOS rat. Taken together, these results indicated that increasing a level of BAT activity and/or increasing an amount of BAT (e.g., by transplantation) reversed irregular estrous cyclicity in the PCOS rat. PCOS Ovarian Phenotypes and Infertility Were Reversed by BAT Transplantation. Histologically, the number of corpora lutea (CL) was decreased and the thickness of the theca cell layer was increased in DHEA+sham and DHEA+Mus groups compared with the control group (Table 2).

TABLE 2

| | | Normal of corpora | Thickness of theca |
|---|---|---|---|
| Group | Total No. | lutea | cell layer (um) |
| Control | 6 | 16 ± 0.58^{a} | 19.34 ± 1.02^{a} |
| DHEA + sham | 6 | 6.67 ± 0.88^{b} | 36.04 ± 0.87^{b} |
| DHEA + Mus | 6 | 5.67 ± 0.33^{b} | 37.26 ± 2.11^{b} |
| DHEA + BAT | 6 | 14.33 ± 1.2^{a} | 19.99 ± 1.03^{a} |

However, abnormal layer of theca cells, mature follicles, and corpus luteum (CL) were observed in the ovary from the DHEA+BAT group (FIG. 5D and Table 2). Previous studies demonstrated that ovarian sympathetic tone was increased in women with PCOS. Tyrosine hydroxylase (TH) is the rate-limiting enzyme in the biosynthesis of norepinephrine (NE), and expression of TH in the ovary is highly restricted to sympathetic nerves. Thus, ovarian tissue sections from four groups were immunostained with an anti-TH antibody to detect sympathetic innervation. A large number of TH-positive sympathetic nerve fibers were found in ovaries from the DHEA+sham and DHEA+Mus groups whereas BAT transplantation significantly reduced the number of TH expressing sympathetic nerve fibers in the ovaries (FIG. 5D).

Consistent with immunostaining results, the expressions of ovarian steroidogenic enzymes, such as P450C17, aromatase, 3β-HSD, 17β-HSD, and STAR, were significantly decreased in the DHEA+sham and DHEA+Mus groups compared with the control group, and increasing a level of BAT activity and/or increasing an amount of BAT (e.g., by administration of adiponectin or BAT transplantation) dramatically reversed their expressions up to normal levels (FIG. 5E). In particular, rats in the DHEA+sham and DHEA+Mus groups were infertile and unable to give birth to a litter; however, BAT transplantation enabled the PCOS rat to deliver a litter (FIG. 5F and Table 3).

TABLE 3

| Group | Total No. | Pregnancy and Parturition |
|---|---|---|
| Control | 9 | 9 |
| DHEA + Sham | 8 | 2 |
| DHEA + Mus | 7 | 2 |
| DHEA + BAT | 7 | 6 |

Collectively, these results indicate that BAT transplantation could significantly reverse infertility in the PCOS rat.

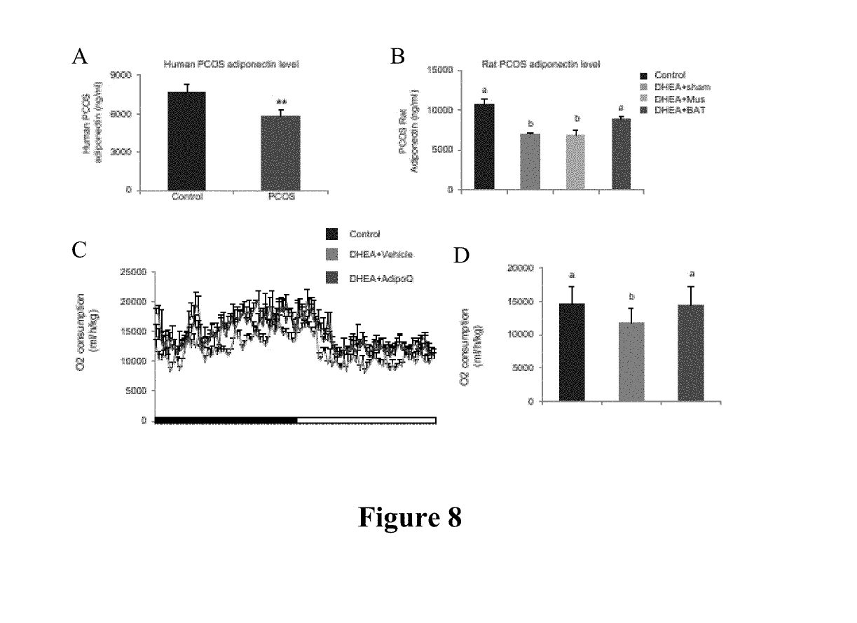

In certain exemplary embodiments increasing a level of BAT activity (e.g., by administration of adiponectin) recapitulates the beneficial effects of increasing the amount of BAT (i.e., BAT transplantation) in a subject in need thereof. It has been reported that the circulating adiponectin level is significantly decreased in both a PCOS patient and PCOS induced rat (FIGS. 8 A and B and Table 4).

TABLE 4

| Variable | PCOS | Control | P |
|---|---|---|---|
| No. | 40 | 40 | |
| Age (y) | 29.35 ± 4.40 | 29.38 ± 4.30 | 0.980 |
| BMI (kg/m²) | 27.36 ± 5.19 | 28.34 ± 4.63 | 0.379 |
| FSH (mIU/mL) | 5.61 ± 1.514 | 6.51 ± 1.42 | <0.001 |
| LH (mIU/mL) | 10.09 ± 5.94 | 4.37 ± 1.90 | 0.008 |
| T (ng/dL) | 45.44 ± 16.04 | 28.90 ± 13.54 | <0.001 |
| E2 (pg/mL) | 44.47 ± 21.07 | 29.82 ± 15.08 | 0.001 |
| 0 min Glu (mmol/L) | 5.58 ± 0.65 | 5.83 ± 0.97 | 0.210 |
| 120 min Glu (mmol/L) | 6.95 ± 2.06 | 6.17 ± 2.05 | 0.101 |
| 0 min INS (mU/L) | 20.65 ± 19.67 | 16.43 ± 6.16 | 0.205 |
| 120 min INS (mIU/L) | 106.48 ± 103.03 | 61.04 ± 63.81 | 0.025 |

Data represent the mean ± SD. BMI, body mass index; E2, estradiol; FSH, follicle-stimulating hormone; Glu, glucose; INS, insulin a; LH, luteinizing hormone; T, testosterone. P < 0.05 compared with the control group; P values were determined by Student's t test.

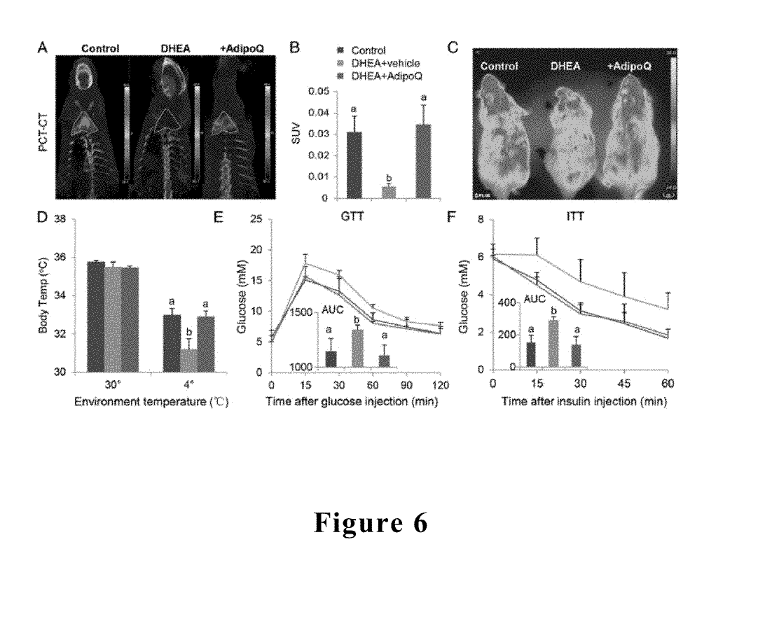

Therefore, it was reasoned that adiponectin might account, at least in part, for the beneficial effects of BAT transplantation in the PCOS rat. To address this question, a PCOS rat was injected daily with recombinant adiponectin protein (10 μg/kg BW) for 20 d. Results from PET-CT (FIGS. 6 A and B), as well as cold-induced thermogenesis (FIGS. 6 C and D), showed that administration of adiponectin in a PCOS rat significantly increased endogenous BAT activity up to the level of the control group. Similar to BAT transplantation, adiponectin treatment also increased energy expenditure and glucose homeostasis in the PCOS rat (FIGS. 6 E and F).

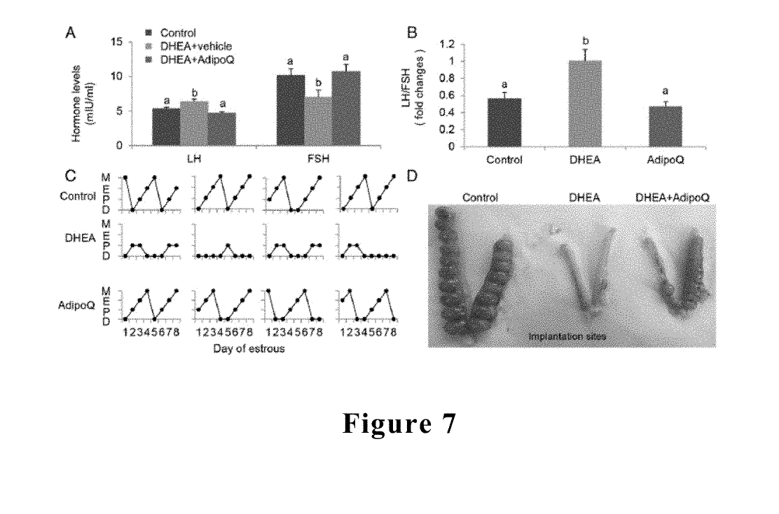

In addition, adiponectin treatment markedly attenuated the plasma LH/FSH ratio that was increased in the DHEA+sham group and it was reversed to a normal level after adiponectin treatment (FIGS. 7 A and B). Interestingly, adiponectin treatment significantly reversed DHEA-induced acyclicity (FIG. 7C and Table 5), ovarian phenotypes (Table 5), and infertility in the PCOS rat (FIG. 7D and Table 5). Data were analyzed by one-way ANOVA with Tukey's post hoc test. n=6 per group. Different lowercase letters indicate significant differences among groups (One-way ANOVA, with Tukey's post hoc test, P<0.05).

TABLE 5

| | | Normal | Abnormal | No. of | Thickness of theca | Pregnancy and |
|---|---|---|---|---|---|---|
| Group | No. | Estrous | Estrous | corpora lutea | cell layer (um) | Parturition |
| Control | 6 | 6 | 0 | 14.33 ± 0.67^{a} | 19.25 ± 1.01^{a} | 6 |
| DHEA + | 6 | 1 | 5 | 6.67 ± 1.2^{b} | 36.54 ± 1.23^{b} | 1 |
| Vehicle | | | | | | |
| DHEA + | 6 | 4 | 2 | 11.67 ± 1.2^{a} | 19.06 ± 0.95^{a} | 4 |
| adipoQ | | | | | | |

Data were analyzed by one-way ANOVA with Tukey's post hoc test. Different lowercase letters indicate significant differences among groups (One-way ANOVA, with Tukey's post hoc test, P < 0.05).

These results highlight that the beneficial effects of BAT transplantation are partly mediated by an elevated circulating adiponectin level.

### Discussion

As shown herein, BAT activity is dramatically decreased in the PCOS rat, however, increasing a level of BAT activity or increasing an amount of BAT (e.g., by transplantation) effectively ameliorated most of the symptoms found in the PCOS rat. In addition, the beneficial effects of BAT transplantation in the PCOS rat were mediated by the increased circulating adiponectin level.

It has been shown that mice neonatally androgenized with testosterone that induces PCOS showed a significant decrease in energy expenditure. It has been speculated that this phenomenon could be due to the BAT hypofunction. In agreement with previous findings, BAT-specific thermogenic gene expression, UCP1, and mitochondrial OXPHOS protein expression and cold-induced thermogenic capacity, which are key factors accounting for the reduction of energy metabolism, were reduced in our PCOS rat (FIG. 1), indicating that the DHEA-induced PCOS rat had a significant defect in energy metabolism and BAT activity.

In parallel, it was also reported that women with PCOS show increased sympathetic tone. Consistently, it was observed that sympathetic innervation, as evidenced by TH staining, was increased in the ovaries of the DHEA-treated PCOS rat (FIG. 2C). Sustained high sympathetic tone causes insensitivity of BAT and later influences disrupted whole-body energy metabolism in PCOS. Taken together, these results suggest that the attenuation of BAT activity might play a significant pathogenic role in PCOS. It has been widely appreciated that women with PCOS show insulin resistance and glucose intolerance. On the other hand, BAT activity is often negatively associated with diabetes status but positively correlated with glucose uptake activity in humans. Recently, we demonstrated that BAT transplantation has a beneficial effect on the prevention and treatment of obesity in the HFD-induced obese mouse, as well as in the genetic obese Ob/Ob mouse. In addition, we showed that BAT transplantation significantly improved glucose homeostasis in both diet-induced obesity and genetic obesity mice models. In agreement with previous results, we observed that DHEA-induced glucose intolerance was significantly reversed by transplantation of BAT, but not muscle (FIGS. 2 E and F). These results again emphasize the important role of BAT in glucose homeostasis.

The remaining question we had was how the transplanted BAT displayed beneficial effects on PCOS. We speculated that the beneficial effects of BAT transplantation might be from activated endogenous BAT that might secrete systemic brown adipose tissue-derived adipokine (batokine). Previously, it was reported that BAT transplantation in obese mice significantly increased the circulating adiponectin level, which is known to be attenuated in women with PCOS. Consistently, we also confirmed that there was a significant reduction of the circulating adiponectin level in both PCOS women and the DHEA-treated rats. As shown herein, the adiponectin level was improved to a normal level after BAT transplantation (FIGS. 8 A and B). In addition, adiponectin administration was shown to improve energy consumption (as measured by O₂ consumption) as seen in FIGS. 8C and D.

Therefore, whether adiponectin administration recapitulates the beneficial effects of BAT transplantation in the PCOS rat was further investigated. After adiponectin treatment, decreased BAT activity, metabolic abnormalities, acyclicity, and abnormal hormonal levels were surprisingly normalized up to normal levels in the PCOS rat. Based on recent publications, BAT also secretes a considerable number of adipokines, such as adiponectin, FGF21, NGF, NRG4, VEGF, and BMPs. It was observed that there was no significant difference of FGF21 or NGF levels between groups (Table 6).

TABLE 6

| | Control | DHEA + Sham | DHEA + Mus | DHEA + BAT |
|---|---|---|---|---|
| Variable | (n = 8) | (n = 9) | (n = 9) | (n = 10) |
| T3 (pmol/L) | 4.48 ± 0.208 | 4.40 ± 0.331 | 4.24 ± 1.151 | 4.45 ± 0.251 |
| T4 (pmol/L) | 32.74 ± 1.454 | 29.84 ± 1.568 | 28.30 ± 1.916 | 29.35 ± 2.542 |
| Estradiol | 31.13 ± 4.689 | 25.78 ± 1.597 | 28.14 ± 5.616 | 24.10 ± 5.177 |
| (pg/mL) | | | | |
| Progesterone | 40.00 ± 1.200 | 37.36 ± 1.621 | 38.61 ± 1.311 | 36.95 ± 1.601 |
| (ng/mL) | | | | |
| Testosterone | 2.41 ± 0.068^{a} | 2.79 ± 0.136^{b} | 2.79 ± 0.077^{b} | 2.35 ± 0.133^{a} |
| (ng/mL) | | | | |
| CHO (mmol/L) | 1.34 ± 0.220 | 1.51 ± 0.116 | 1.33 ± 0.107 | 1.06 ± 0.144 |
| TG (mmol/L) | 0.28 ± 0.032 | 0.40 ± 0.044 | 0.38 ± 0.043 | 0.28 ± 0.044 |
| HDL (mmol/L) | 1.55 ± 0.174^{a} | 1.34 ± 0.022^{b} | 1.11 ± 0.079^{b} | 1.15 ± 0.072^{a} |
| LDL (mmol/L) | 0.12 ± 0.016 | 0.14 ± 0.014 | 0.13 ± 0.017 | 0.12 ± 0.012 |
| NGF (pg/mL) | 47.11 ± 5.658 | 48.42 ± 5.860 | 49.33 ± 8.420 | 55.35 ± 7.548 |
| FGF21 (pg/mL) | 45.50 ± 7.910 | 47.12 ± 8.100 | 50.17 ± 11.078 | 56.79 ± 10.726 |

Data were analyzed by one-way ANOVA with Tukey's post hoc test. Different lowercase letters indicate significant differences among groups (One-way ANOVA, with Tukey's post hoc test, P < 0.05).
CHO, cholesterol;
FGF21, fibroblast growth factor 21;
HDL, high-density lipoprotein;
LDL, low-density lipoprotein;
NGF, nerve growth factor;
T3, triiodothyronine;
T4, thyroxine;
TG, triglyceride

It has been suggested that BAT secretes systemic mediators that could regulate in whole-body glucose homeostasis. It should be noted that we do not exclude other factors mentioned above that may be involved in the beneficial effects of BAT transplantation in the PCOS rat model. However, in our hands, we observed that adiponectin alone was enough to recapitulate the beneficial effects of BAT transplantation in the PCOS rat. Other mechanisms behind the adiponectin effect for the treatment of PCOS would be necessary to be revealed in the near future. Taken together, our findings highlight that systemic adiponectin treatment significantly improves PCOS phenotypes in an animal model.

In certain exemplary embodiments, Applicants have demonstrated that BAT transplantation (and thereby increased BAT activity) significantly improves PCOS phenotypes (and symptoms thereof), including disrupted energy metabolism, acyclicity, and infertility. In addition, these beneficial effects of BAT transplantation were at least in part mediated by systemic adiponectin. In certain exemplary embodiments, BAT transplantation may be mimicked by, for example, administration of batokines or drugs that enhance BAT activity will be alternative strategies for the treatment of PCOS.

Although the present disclosure has been described with reference to specific embodiments, it should be understood that the limitations of the described embodiments are provided merely for purpose of illustration and are not intended to limit the present invention and associated general inventive concepts. Instead, the scope of the present invention is defined by the appended claims, and all variations and equivalents that fall within the range of the claims are intended to be embraced therein. Thus, other embodiments than the specific exemplary ones described herein are equally possible within the scope of these appended claims.

### REFERENCES

1. March W A, et al. (2010) The prevalence of polycystic ovary syndrome in a community sample assessed under contrasting diagnostic criteria. Hum Reprod 25(2):544-551.
2. Azziz R, et al. (2004) The prevalence and features of the polycystic ovary syndrome in an unselected population. J Clin Endocrinol Metab 89(6):2745-2749.
3. Muscogiuri G, Colao A, Orio F (2015) Insulin-mediated diseases: Adrenal mass and polycystic ovary syndrome. Trends Endocrinol Metab 26(10):512-514.
4. Moran L J, Strauss B J, Teede H J (2011) Diabetes risk score in the diagnostic categories of polycystic ovary syndrome. Fertil Steril 95(5):1742-1748.
5. Lindholm A, Andersson L, Eliasson M, Bixo M, Sundström-Poromaa I (2008) Prevalence of symptoms associated with polycystic ovary syndrome. Int J Gynaecol Obstet 102(1): 39-43.
6. Spritzer P M (2014) Polycystic ovary syndrome: Reviewing diagnosis and management of metabolic disturbances. Arq Bras Endocrinol Metabol 58(2):182-187.
7. Stepto N K, et al. (2013) Women with polycystic ovary syndrome have intrinsic insulin resistance on euglycaemic-hyperinsulaemic clamp. Hum Reprod 28(3):777-784.
8. Goodarzi M O, Dumesic D A, Chazenbalk G, Azziz R (2011) Polycystic ovary syndrome: Etiology, pathogenesis and diagnosis. Nat Rev Endocrinol 7(4):219-231.
9. Ehrmann D A (2005) Polycystic ovary syndrome. N Engl J Med 352(12):1223-1236.
10. Legro R S, et al.; Endocrine Society (2013) Diagnosis and treatment of polycystic ovary syndrome: An Endocrine Society clinical practice guideline. J Clin Endocrinol Metab 98(12):4565-4592.
11. Ozegowska K E, Pawelczyk L A (2015) The role of insulin and selected adipocytokines in patients with polycystic ovary syndrome (PCOS): A literature review. Ginekol Pol 86(4):300-304.
12. Villa J, Pratley R E (2011) Adipose tissue dysfunction in polycystic ovary syndrome. Curr Diab Rep 11(3):179-184.
13. Cannon B, Nedergaard J (2004) Brown adipose tissue: Function and physiological significance. Physiol Rev 84(1):277-359.
14. Virtanen K A, et al. (2009) Functional brown adipose tissue in healthy adults. N Engl J Med 360(15):1518-1525.
15. Cypess A M, et al. (2009) Identification and importance of brown adipose tissue in adult humans. N Engl J Med 360(15):1509-1517.
16. Wang G X, Zhao X Y, Lin J D (2015) The brown fat secretome: Metabolic functions beyond thermogenesis. Trends Endocrinol Metab 26(5):231-237.
17. Liu X, et al. (2015) Brown adipose tissue transplantation reverses obesity in Ob/Ob mice. Endocrinology 156(7):2461-2469.
18. Liu X, et al. (2013) Brown adipose tissue transplantation improves whole-body energy metabolism. Cell Res 23(6):851-854.
19. Stanford K I, et al. (2013) Brown adipose tissue regulates glucose homeostasis and insulin sensitivity. J Clin Invest 123(1):215-223.
20. Ehrmann D A, Barnes R B, Rosenfield R L, Cavaghan M K, Imperial J (1999) Prevalence of impaired glucose tolerance and diabetes in women with polycystic ovary syndrome. Diabetes Care 22(1):141-146.
21. Betz M J, Enerbäck S (2015) Human brown adipose tissue: What we have learned so far. Diabetes 64(7):2352-2360.
22. Shen Y, et al. (2014) Recent advances in brown adipose tissue biology. Chin Sci Bull 59(31):4030-4040.
23. Evans R M, Barish G D, Wang Y X (2004) PPARs and the complex journey to obesity. Nat Med 10(4):355-361.
24. de Jesus L A, et al. (2001) The type 2 iodothyronine deiodinase is essential for adaptive thermogenesis in brown adipose tissue. J Clin Invest 108(9):1379-1385.
25. Robinson S, et al. (1992) Postprandial thermogenesis is reduced in polycystic ovary syndrome and is associated with increased insulin resistance. Clin Endocrinol (Oxf) 36(6):537-543.
26. Lansdown A, Rees D A (2012) The sympathetic nervous system in polycystic ovary syndrome: A novel therapeutic target? Clin Endocrinol (Oxf) 77(6):791-801.
27. Dissen G A, et al. (2009) Excessive ovarian production of nerve growth factor facilitates development of cystic ovarian morphology in mice and is a feature of polycystic ovarian syndrome in humans. Endocrinology 150(6):2906-2914.
28. Mannerås-Holm L, et al. (2011) Adipose tissue has aberrant morphology and function in PCOS: Enlarged adipocytes and low serum adiponectin, but not circulating sex steroids, are strongly associated with insulin resistance. J Clin Endocrinol Metab 96(2): E304-E311.
29. Nohara K, et al. (2011) Early-life exposure to testosterone programs the hypothalamic melanocortin system. Endocrinology 152(4):1661-1669.
30. Nohara K, et al. (2013) Developmental androgen excess programs sympathetic tone and adipose tissue dysfunction and predisposes to a cardiometabolic syndrome in female mice. Am J Physiol Endocrinol Metab 304(12):E1321-E1330.
31. Sverrisdóttir Y B, Mogren T, Kataoka J, Janson P O, Stener-Victorin E (2008) Is polycystic ovary syndrome associated with high sympathetic nerve activity and size at birth? Am J Physiol Endocrinol Metab 294(3):E576-E581.
32. Legro R S (2003) Polycystic ovary syndrome and cardiovascular disease: A premature association? Endocr Rev 24(3):302-312.
33. Ouellet V, et al. (2011) Outdoor temperature, age, sex, body mass index, and diabetic status determine the prevalence, mass, and glucose-uptake activity of 18F-FDG-detected BAT in humans. J Clin Endocrinol Metab 96(1):192-199.
34. Escobar-Morreale H F, San Millán J L (2007) Abdominal adiposity and the polycystic ovary syndrome. Trends Endocrinol Metab 18(7):266-272.
35. Keipert S, et al. (2015) Genetic disruption of uncoupling protein 1 in mice renders brown adipose tissue a significant source of FGF21 secretion. Mol Metab 4(7): 537-542.
36. Gunawardana S C, Piston D W (2012) Reversal of type 1 diabetes in mice by brown adipose tissue transplant. Diabetes 61(3):674-682.
37. Rotterdam ESHRE/ASRM-Sponsored PCOS Consensus Workshop Group (2004) Revised 2003 consensus on diagnostic criteria and long-term health risks related to polycystic ovary syndrome (PCOS). Hum Reprod 19(1):41-47.
38. Caldwell A S, et al. (2014) Characterization of reproductive, metabolic, and endocrine features of polycystic ovary syndrome in female hyperandrogenic mouse models. Endocrinology 155(8):3146-3159.
39. Chi Q S, Wang D H (2011) Thermal physiology and energetics in male desert hamsters (Phodopus roborovskii) during cold acclimation. J Comp Physiol B 181(1):91-103

1. A method for the treatment, delay of progression, or prevention of at least one symptom of polycyctic ovary syndrome, the method comprising increasing brown adipose tissue activity in a subject in need thereof, wherein at least one symptom of polycystic ovary syndrome is reduced.

2. The method of claim 1, wherein the brown adipose tissue activity is increased by increasing an amount of brown adipose tissue in the subject.

3. The method of claim 2, wherein the amount of brown adipose tissue is increased by transplanting brown adipose tissue into the subject.

4. The method of claim 1, wherein the brown adipose tissue activity is increased by administration of a batokine.

5. The method of claim 1, wherein the brown adipose tissue activity is increased by administration of adiponectin.

6. The method of claim 1, wherein the symptom is selected from the group comprising: an imbalance in the sex hormones estrogen and progesterone, reduced energy expenditure, obesity, dyslipidemia, type 2 diabetes, cardiovascular disease, infertility, insulin resistance, androgen excess, anovulation, irregular menstruation, polycystic ovary appearance on ultrasound, enlarged ovaries, and irregular hair growth.

7. The method of claim 1, wherein the symptom is infertility.

8. The method of claim 1, wherein the symptom is an imbalance in the sex hormones estrogen and progesterone.

9. The method of claim 1, wherein the symptom is insulin resistance.

10. A method for the treatment, delay of progression, or prevention of polycystic ovary syndrome, the method comprising increasing brown adipose tissue activity in a subject in need thereof, wherein at least one symptom of polycystic ovary syndrome is reduced.

11. The method of claim 10, wherein the brown adipose tissue activity is increased by increasing an amount of brown adipose tissue in the subject.

12. The method of claim 11, wherein the amount of brown adipose tissue is increased by transplanting brown adipose tissue into the subject.

13. A method for the treatment, delay of progression, or prevention of polycystic ovary syndrome, the method comprising administration of adiponectin to a subject in need thereof, wherein at least one symptom of polycystic ovary syndrome is reduced.
